(19) United States
(12) Patent Application Publication
(10) Pub. No.: US 2025/0313529 A1
(43) Pub. Date: Oct. 9, 2025

(54) SYNTHESIS OF SMALL MOLECULE AGONISTS OF NEUROPTROPHIN

(71) Applicant: Oculis Operations Sàrl, Lausanne (CH)

(72) Inventors: Jón Freyr Egilsson, Hafnarfjordur (IS); Gudrun Marta Asgrimsdottir, Reykjavik (IS)

(73) Assignee: Oculis Operations Sàrl, Lausanne (CH)

(21) Appl. No.: 19/244,594

(22) Filed: Jun. 20, 2025

Related U.S. Application Data
(63) Continuation of application No. PCT/EP2023/087549, filed on Dec. 22, 2023.

(30) Foreign Application Priority Data
Dec. 22, 2022 (EP) 22215831.3

(51) Int. Cl. C07D 207/27; C07C 231/02; C07C 233/47; C07C 233/51

(52) U.S. Cl. CPC C07D 207/27; C07C 231/02; C07C 233/47; C07C 233/51

(57) ABSTRACT

The disclosure concerns a process for synthesizing a compound of formula FI:

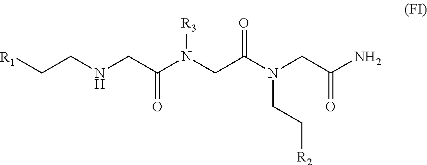

wherein:
R₁ is phenyl substituted with halogen or trifluoromethyl, and further optionally substituted with one or two substituents selected from the group consisting of halogen, (C₁-C₆)alkyl, (C₁-C₆)alkoxy, and halo(C₁-C₆)alkyl; or alternatively R₁ is pyrrolidin-1-yl;
R₂ is 2-oxo-pyrrolidin-1-ylmethyl or sulfamoylphenyl; and
R₃ is chosen from propyl, 1-methylethyl, butyl, 2-methylpropyl, pentyl, 1-methyl-butyl, 2-methylbutyl, hexyl, 4-methylpentyl, 3-methylpentyl, 2-methylpentyl, and 1-methylpentyl.

### CROSS-REFERENCE TO RELATED APPLICATIONS

This application is a continuation of International Application No. PCT/EP2023/087549 filed in the European Receiving Office on Dec. 22, 2023, which claims the benefit of European Provisional Application No. 22215831.3, filed on Dec. 22, 2022. The entirety of each of these applications is hereby incorporated by reference herein for all purposes.

### FIELD OF THE INVENTION

This disclosure pertains to the field of therapeutics for neurological, psychiatric disorders, and ageing. In particular, this disclosure relates to a synthesis route of small molecule agonists of neurotrophin (Nerve Growth Factor (NGF) or Brain-Derived Neurotrophic Factor (BDNF)), more specifically small molecules of formula FI:

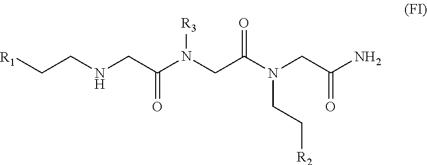

### BACKGROUND

A synthesis route for compounds of formula FI is known. For instance, Masip et al. (Med. Chem. 13 (2005)1929 (doi:/j.bmc.2005.01.024) discloses a solid phase synthesis using positional scanning format and submonomer strategy. This synthesis route comprises 7 steps before a final step of cleavage to recover the targeted molecule from the solid support (Rink amide resin), as shown below:

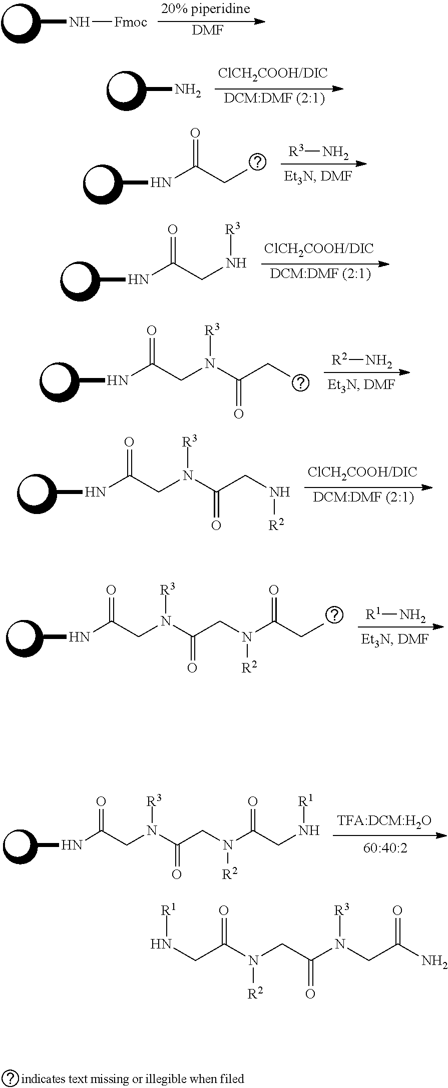

The first step of this synthesis route is the deprotection of the amine function of the solid support. The six following steps are a repetition of a sequence of two steps, namely an acylation immediately followed by an amination. Therefore, in this synthesis route, molecules are grown sequentially by using appropriate primary amines regarding the targeted molecule.

Although this synthesis step may be satisfactory in certain conditions, it would be beneficial to propose a synthesis route without using a solid support, in particular which would be more suitable for large-scale synthesis for industrial manufacturing.

### SUMMARY OF THE INVENTION

It is proposed a process for synthesizing a compound of formula FI:

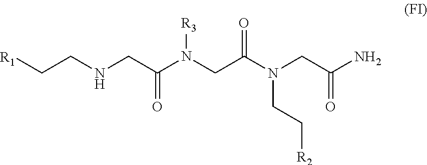

wherein:
R₁ is phenyl substituted with halogen or trifluoromethyl, and further optionally substituted with one or two substituents selected from the group consisting of halogen, (C₁-C₆)alkyl, (C₁-C₆)alkoxy, and halo(C₁-C₆)alkyl; or alternatively R₁ is pyrrolidin-1-yl;
R₂ is 2-oxo-pyrrolidin-1-ylmethyl or sulfamoylphenyl; and
R₃ is chosen from propyl, 1-methylethyl, butyl, 2-methylpropyl, pentyl, 1-methyl-butyl, 2-methylbutyl, hexyl, 4-methylpentyl, 3-methylpentyl, 2-methylpentyl, and 1-methylpentyl; comprising the steps of:
   (S1) Providing a compound CS1 of formula FCS1:

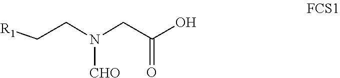

(S2) Providing a compound CS2 of formula FCS2:

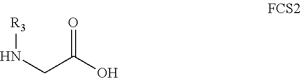

(S3) Providing a compound CS3 of formula FCS3:

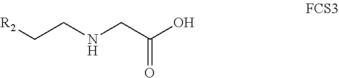

(S4) Reacting CS1 with CS2 in an organic solvent resulting in a compound CS4 of formula FCS4:

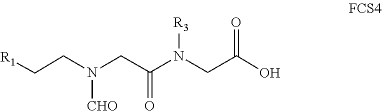

(S5) Reacting CS3 with CS4 in an organic solvent resulting in a compound CS5 of formula FCS5:

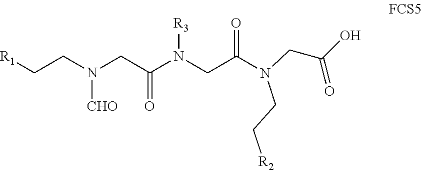

(S6) Reacting CS5 with an acid in an alcoholic solvent resulting in a compound CS6 of formula FCS6:

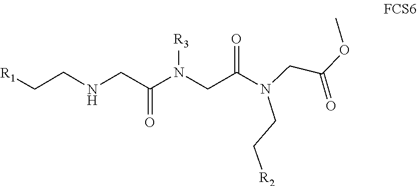

(S7) Reacting CS6 with ammonia in an organic solvent.

### BRIEF DESCRIPTION OF THE FIGURES

FIG. 1 represents a synthesis route according to the disclosure of compound of formula FI wherein R₁ is 2-fluorophenyl, R₂ is methylpyrolidin-2-one and R₃ is isobutyl.

FIG. 2 shows the ¹HNMR spectra in CDCl₃ of 2-{N-[2-(2-fluorophenyl)ethyl]formamido}acetic acid.

FIG. 3 shows the ¹HNMR spectra of 2-[(2-methylpropyl)amino]acetic acid hydrochloride in DMSO.

FIG. 4 shows the ¹HNMR spectra of 2-{[3-(2-oxopyrrolidin-1yl)propyl]amino}acetic acid hydrochloride in DMSO.

FIG. 5 shows the ¹HNMR spectra of N-(carbamoylmethyl)-2-(2-{[2-(2-fluorophenyl)ethyl]amino}-N-(2-methylpropyl)acetamido)-N-[3-(2-oxopyrrolidin-1yl)propyl]acetamide in CD₃OD.

### DETAILED DESCRIPTION OF THE INVENTION

As mentioned above, the disclosure concerns a process for synthesizing a compound of formula FI:

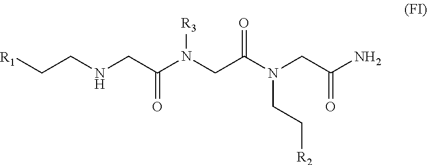

wherein:
R₁ is phenyl substituted with halogen or trifluoromethyl, and further optionally substituted with one or two substituents selected from the group consisting of halogen, (C₁-C₆)alkyl, (C₁-C₆)alkoxy, and halo(C₁-C₆)alkyl; or alternatively R₁ is pyrrolidin-1-yl;
R₂ is 2-oxo-pyrrolidin-1-ylmethyl or sulfamoylphenyl; and
R₃ is chosen from propyl, 1-methylethyl, butyl, 2-methylpropyl, pentyl, 1-methyl-butyl, 2-methylbutyl, hexyl, 4-methylpentyl, 3-methylpentyl, 2-methylpentyl, and 1-methylpentyl; comprising the steps of:
   (S1) Providing a compound CS1 of formula FCS1:

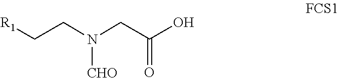

(S2) Providing a compound CS2 of formula FCS2:

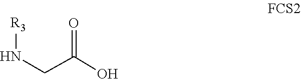

(S3) Providing a compound CS3 of formula FCS3:

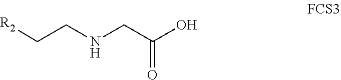

(S4) Reacting CS1 with CS2 in an organic solvent resulting in a compound CS4 of formula FCS4:

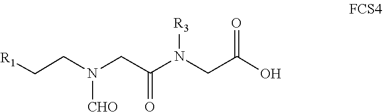

(S5) Reacting CS3 with CS4 in an organic solvent resulting in a compound CS5 of formula FCS5:

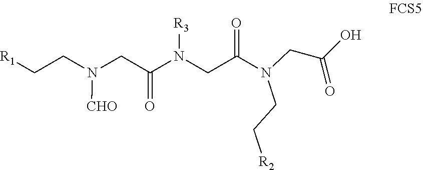

(S6) Reacting CS5 with an acid in an alcoholic solvent resulting in a compound CS6 of formula FCS6:

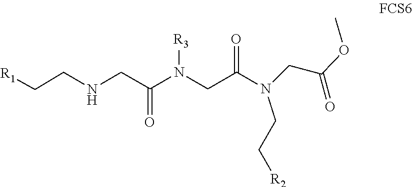

(S7) Reacting CS6 with ammonia in an organic solvent.

### The Step (S1)

According to the present disclosure, step (S1) consists of providing a compound of formula FCS1. To the knowledge of the applicant, at the time of the present disclosure, compounds of formula FCS1 are not commercially available.

CS1 can be obtained by reacting glyoxylic acid with an amine of formula NH₂—CH₂—CH₂—R₁. Accordingly, in an embodiment, step (S1) is carried out by reacting glyoxylic acid with an amine of formula NH₂—CH₂—CH₂—R₁ in water at a temperature between 65° C. and 90° C., preferably between 70° C. and 85° C. and more preferably about 80° C.

In a specific embodiment, step (S1) is carried out at a ratio glyoxylic acid to amine of about 2.05, for instance between 2.0 and 2.25.

According to the present disclosure, R₁ is phenyl substituted with halogen or trifluoromethyl, and further optionally substituted with one or two substituents selected from the group consisting of halogen, (C₁-C₆)alkyl, (C₁-C₆)alkoxy, and halo(C₁-C₆)alkyl; or alternatively R₁ is pyrrolidin-1-yl.

In a preferred embodiment R₁ is 2-fluorophenyl.

### The Step (S2)

According to the present disclosure, step (S2) consists of providing a compound of formula FCS2. To the knowledge of the applicant, at the time of the present disclosure, compounds of formula FCS2 have already been disclosed.

However, CS2 can be obtained by reacting glyoxylic acid with an amine of formula NH₂—R₃. Accordingly, in an embodiment, step (S2) is carried out by reacting glyoxylic acid with an amine of formula NH₂—R₃ in water at a temperature between 65° C. and 90° C., preferably between 70° C. and 85° C. and more preferably about 80° C., and then concentrated hydrochloric acid (HCl) is added at a temperature of 80° C. or above, preferably 90° C. or above and more preferably of 100° C.

In an embodiment concentrated HCl is an aqueous solution at 35% HCl.

The ratio HCl to amine of formula NH₂—R₃ is comprised between 1.05 and 5, preferably between 1.5 and 4, more preferably between 2 and 3.

According to the present disclosure, R₃ is chosen from propyl, 1-methylethyl, butyl, 2-methylpropyl, pentyl, 1-methyl-butyl, 2-methylbutyl, hexyl, 4-methylpentyl, 3-methylpentyl, 2-methylpentyl, and 1-methylpentyl.

In a preferred embodiment, R₃ is 2-methylpropyl.

### The Step (S3)

According to the present disclosure, step (S3) consists of providing a compound of formula FCS3. To the knowledge of the applicant, at the time of the present disclosure, compounds of formula FCS3 are not commercially available.

CS3 can be obtained by reacting glyoxylic acid with an amine of formula NH₂—CH₂—CH₂—R₂. Accordingly, in an embodiment, step (S3) is carried out by reacting glyoxylic acid with an amine of formula NH₂—CH₂—CH₂—R₂ in water at a temperature between 65° C. and 90° C., preferably between 70° C. and 85° C. and more preferably about 80° C., and then concentrated HCl is added at a temperature of 80° C. or above, preferably 90° C. or above and more preferably of 100° C.

In an embodiment concentrated HCl is an aqueous solution at 35% HCl.

The ratio HCl to amine of formula NH₂— R₃ is comprised between 1.05 and 5, preferably between 1.5 and 4, more preferably between 2 and 3.

According to the present disclosure, R₂ is 2-oxo-pyrrolidin-1-ylmethyl or sulfamoylphenyl.

In a preferred embodiment, R₂ is 2-oxo-pyrrolidin-1-ylmethyl.

### The Step (S4)

According to the present disclosure, step (S4) consists of synthesizing a compound of formula FCS4. To the knowledge of the applicant, at the time of the present disclosure, compounds of formula FCS4 are not commercially available.

CS4 can be obtained by reacting CS1 with CS2 in an organic solvent. Accordingly, in an embodiment, step (S4) is carried out by reacting CS1 with CS2 at a temperature starting at 0° C. to room temperature between 20° C. and 25° C. in the presence of 2,3,4,6,7,8,9,10-octahydropyrimido[1,2-a]azepine, also known as 1,8-Diazabicyclo[5.4.0]undec-7-ene (DBU) and carbonyldiimidazole.

The organic solvent in step (S4) may be selected from the group consisting of dimethylformamide and acetonitrile . . . .

### The Step (S5)

According to the present disclosure, step (S5) consists of synthesizing a compound of formula FCS5. To the knowledge of the applicant, at the time of the present disclosure, compounds of formula FCS5 are not commercially available.

CS5 can be obtained by reacting CS3 with CS4 in an organic solvent. Accordingly, in an embodiment, step (S5) is carried out at room temperature between 20° C. and 25° in the presence of 2,3,4,6,7,8,9,10-octahydropyrimido[1,2-a]azepine, also known as 1,8-Diazabicyclo[5.4.0]undec-7-ene (DBU) and carbonyldiimidazole.

The organic solvent in step (S4) may be selected from the group consisting of dimethylformamide, acetonitrile and dimethylsulfoxide.

### The Step (S6)

According to the present disclosure, step (S6) consists of synthesizing a compound of formula FCS6. To the knowledge of the applicant, at the time of the present disclosure, compounds of formula FCS6 are not commercially available.

CS6 can be obtained by reacting CS5 with an acid in an alcoholic solvent, preferably with H₂SO₄ in methanol.

In an embodiment, step (S6) is carried out at reflux.

### The Step (S7)

Step (S7) is the final step of the synthesis of compounds of formula FI according to the present disclosure. In this final step, CS6 reacts with ammonia.

In an embodiment, step (S7) is carried out at a temperature between −30° C. and −50° C., preferably between −35° C. and −45° C. and more preferably of −40° C.

In step (S7), ammonia could be in a liquid form, for instance in an aqueous or alcoholic, e.g. methanol solution or in a gaseous form, gaseous form being preferred.

In an embodiment, compound of formula FI is obtained with less than 0.3% of each individual impurity at the end of step (S7).

### The Compound of Formula FI

According to the present disclosure, compound of formula FI is the following:

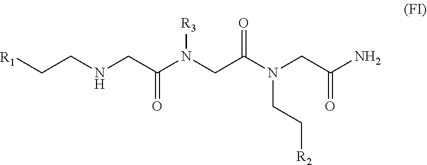

wherein:
R₁ is phenyl substituted with halogen or trifluoromethyl, and further optionally substituted with one or two substituents selected from the group consisting of halogen, (C₁-C₆)alkyl, (C₁-C₆)alkoxy, and halo(C₁-C₆)alkyl; or alternatively R₁ is pyrrolidin-1-yl;
R₂ is 2-oxo-pyrrolidin-1-ylmethyl or sulfamoylphenyl; and
R₃ is chosen from propyl, 1-methylethyl, butyl, 2-methylpropyl, pentyl, 1-methyl-butyl, 2-methylbutyl, hexyl, 4-methylpentyl, 3-methylpentyl, 2-methylpentyl, and 1-methylpentyl.

When R₁ is (C₁-C₆)alkyl, it may be methyl, ethyl, propyl, butyl, 2-methylpropyl, pentyl, 2 methylbutyl, 2,2-dimethylpropyl, hexyl, 2-methylpentyl, 3-methylpentyl, 2,2-dimethylbutyl, 2,3-dimethylbutyl, R₁ being linked by any carbon.

When R₁ is (C₁-C₆)alkoxy, it may be —O—R₄, wherein R₄ may be methyl, ethyl, propyl, butyl, 2-methylpropyl, pentyl, 2 methylbutyl, 2,2-dimethylpropyl, hexyl, 2-methylpentyl, 3-methylpentyl, 2,2-dimethylbutyl, 2,3-dimethylbutyl, R₄ being linked by any carbon to the oxygen, and R₁ being linked by the oxygen.

When R₁ is halo(C₁-C₆)alkyl, it may be —X—R₄, wherein X is a halogen atom, preferably selected from fluorine, chlorine and bromine, wherein R₄ may be methyl, ethyl, propyl, butyl, 2-methylpropyl, pentyl, 2 methylbutyl, 2,2-dimethylpropyl, hexyl, 2-methylpentyl, 3-methylpentyl, 2,2-dimethylbutyl, 2,3-dimethylbutyl, R₄ being linked by any carbon to the halogen atom, and R₁ being linked by the oxygen.

In a preferred embodiment, R₁ is 2-fluorophenyl, R₂ is 2-oxo-pyrrolidin-1-ylmethyl and R₃ is 2-methylpropyl. Thus, in a preferred embodiment, compound of formula FI is the following:

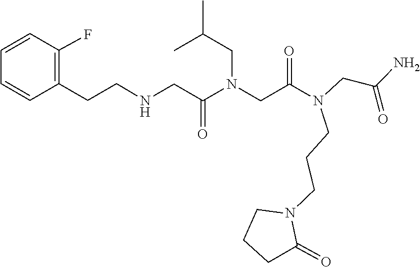 [SMILES: CC(C)CN(CC(=O)N(CCCN1CCCC1=O)CC(N)=O)C(=O)CNCCc1ccccc1F]

### The Compound CS1

Step (S1) of the process according to the present disclosure provides a compound of formula FCS1:

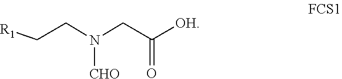

In a preferred embodiment, R₁ is 2-fluorophenyl. Thus, in a preferred embodiment, compound of formula FCS1 is the following:

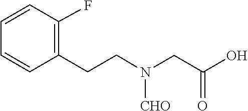 [SMILES: O=CN(CCc1ccccc1F)CC(=O)O]

### The Compound CS2

Step (S2) of the process according to the present disclosure provides a compound of formula FCS2:

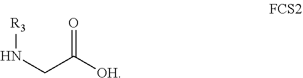

In a preferred embodiment, R₃ is 2-methylpropyl. Thus, in a preferred embodiment, compound of formula FCS2 is the following:

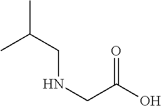

### The Compound CS3

Step (S3) of the process according to the present disclosure provides a compound of formula FCS3:

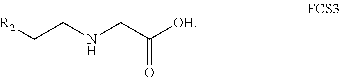

In a preferred embodiment, R₂ is 2-oxo-pyrrolidin-1-ylmethyl. Thus, in a preferred embodiment, compound of formula FCS3 is the following:

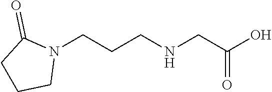 [SMILES: O=C(O)CNCCCN1CCCC1=O]

### The Compound CS4

Step (S4) of the process according to the present disclosure provides a compound of formula FCS4:

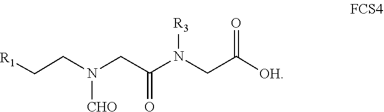

In a preferred embodiment, R₁ is 2-fluorophenyl and R₃ is 2-methylpropyl. Thus, in a preferred embodiment, compound of formula FCS4 is the following:

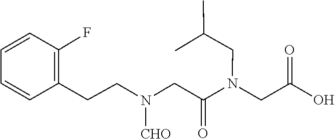 [SMILES: CC(C)CN(CC(=O)O)C(=O)CN(C=O)CCc1ccccc1F]

### The Compound CS5

Step (S5) of the process according to the present disclosure provides a compound of formula FCS5:

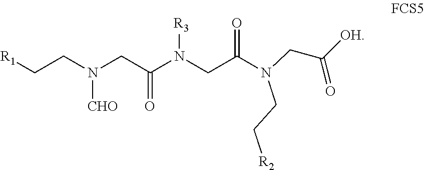

In a preferred embodiment, R₁ is 2-fluorophenyl, R₂ is 2-oxo-pyrrolidin-1-ylmethyl and R₃ is 2-methylpropyl. Thus, in a preferred embodiment, compound of formula FCS5 is the following:

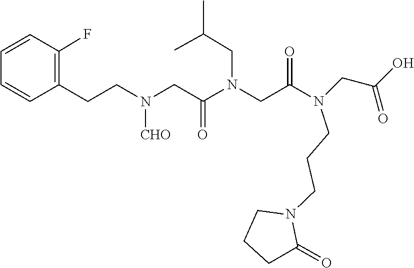 [SMILES: CC(C)CN(CC(=O)N(CCCN1CCCC1=O)CC(=O)O)C(=O)CN(C=O)CCc1ccccc1F]

### The Compound CS6

Step (S6) of the process according to the present disclosure provides a compound of formula FCS6:

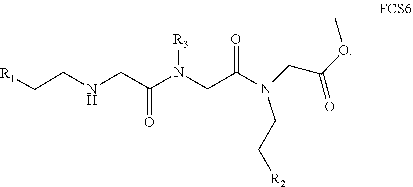

In a preferred embodiment, R₁ is 2-fluorophenyl, R₂ is 2-oxo-pyrrolidin-1-ylmethyl and R₃ is 2-methylpropyl. Thus, in a preferred embodiment, compound of formula FCS6 is the following:

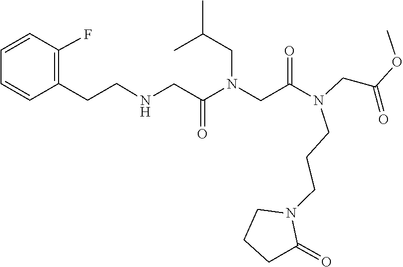 [SMILES: COC(=O)CN(CCCN1CCCC1=O)C(=O)CN(CC(C)C)C(=O)CNCCc1ccccc1F]

### Pharmaceutical Compositions

The disclosure also concerns pharmaceutical compositions comprising the compound of formula FI obtained by the synthesis method described above, and optionally one or more pharmaceutically acceptable excipient(s).

In an embodiment, the pharmaceutical composition comprises less than 0.3% of each individual impurity.

### Example

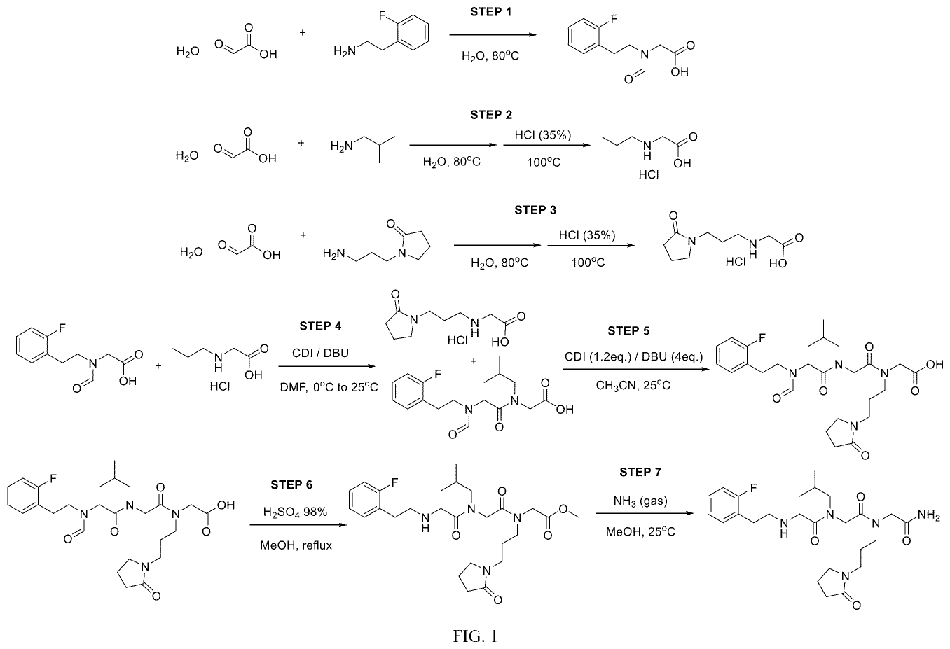

### Synthesis of N-(carbamoylmethyl)-2-(2-{[2-(2-fluorophenyl)ethyl]amino}-N-(2-methylpropyl)acetamido)-N-[3-(2-oxopyrrolidin-1yl)propyl]acetamide (FIG. 1)

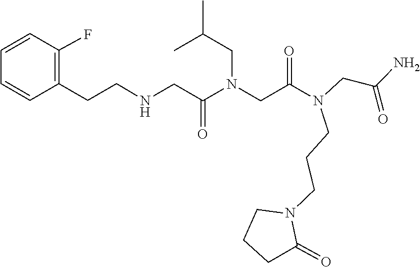 [SMILES: CC(C)CN(CC(=O)N(CCCN1CCCC1=O)CC(N)=O)C(=O)CNCCc1ccccc1F]

### Step (S1)

A solution of Glyoxylic acid (50% wt in water) (8939 ml, 81.02 mol) was added to a suspension of 2-fluorophenethylamine (5500 g, 39.52 mol) in water (27.5 L) and was stirred at 80° C. (internal temp). Cream solid appeared after amine addition.

The solid, formed previously, became to acquire gummy consistence. Afterwards the reaction was heated at 80° C. (internal temp) for another 3 hours.

Liquid Chromatography-Mass spectroscopy (LC-MS) showed total conversion to the desired product.

Reaction was cooled to 5° C. (2 hours) and solid formed was filtered and washed with cold water (5° C.) (5000 ml) and dried at 50° C. under vacuo till constant weight.

Solid were triturated in 5000 ml of isopropanol (iPrOH) and filtered after 30 min. Solid was dried at 50° C. under vacuo till constant weight. [0067]6410 g (72%) of 2-{N-[2-(2-fluorophenyl)ethyl]formamido}acetic acid as a white off solid were obtained.

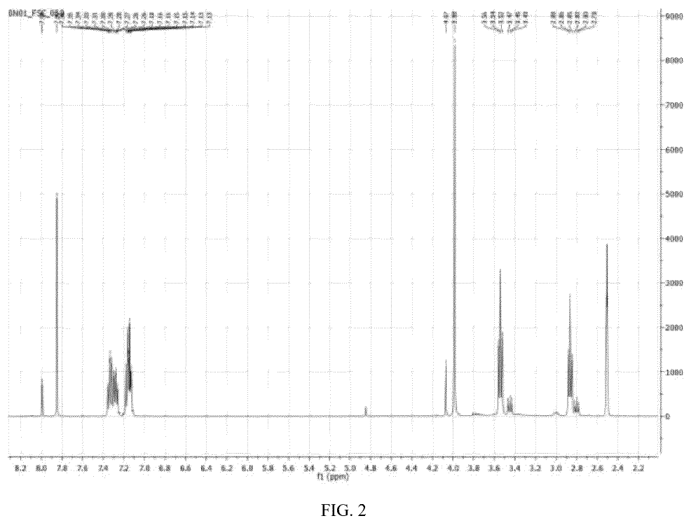

FIG. 2 shows the 1HNMR spectra in CDCl₃ of 2-{N-[2-(2-fluorophenyl)ethyl]formamido}acetic acid.

### Step (S2)

To a solution of isobutylamine (5400 ml, 54.34 mol) in water (16 L) was added Glyoxylic acid (50% wt in water) (12.3 L, 116.8 mol) keeping internal temp below 65° C. (1 h 30 min). A large quantity of bubbles appeared during the addition (CO2 emission).

Then solution was stirred at 82-86° C. (internal temp) for 16 hours.

Solution turned to a dark brown liquid after 2 hours.

After 16 hours the reaction was cooled to 65° C. and 12M HCl (4989 ml, 57.87 mol) were added in one portion and this solution was heated for another 24 hours at 82-86° C. (internal temp).

After 24 hours, water was removed and to the residue was added 5 L of acetonitrile. This solvent was eliminated to remove water traces.

Crude obtained was solved in 25 L of Acetonitrile and heated at 80° C. till total solution. Then the solution was cooled to 25° C. and seeded with desired product (lot BN01_FSC_075). After 16 hours, solid was filtered.

Solid obtained were dried in vacuo at 70° C. till constant weight.

5829 g (64%) of 2-[(2-methylpropyl)amino]acetic acid hydrochloride as a white flakes were obtained.

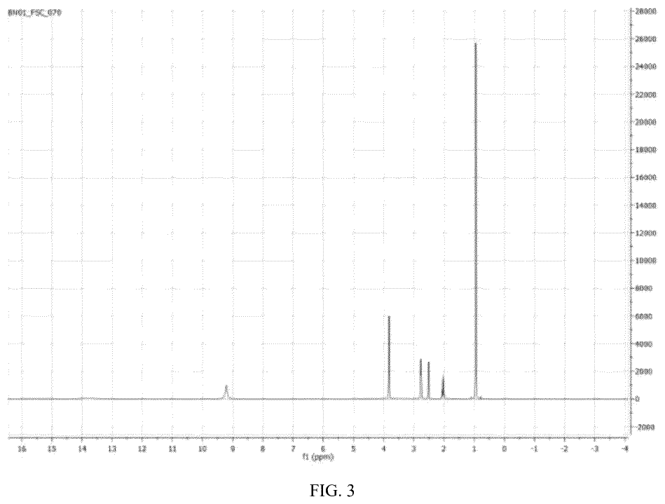

FIG. 3 shows the 4HNMR spectra of 2-[(2-methylpropyl)amino]acetic acid hydrochloride in DMSO.

### Step (S3)

A solution of 1-(3-aminopropyl)pyrrolidin-2-one (4000 g, 28.13 mol) and Glyoxilic acid monohydrate (5821 g, 57.8 mol) in water (30 L) was heated at 85° C. for 12 hours. Then HCl (35%) (4 L, 33.9 mol) was added and the reaction was heated at 90° C. for another 4 hours. Solvent was removed and 2000 ml of acetonitrile were added to eliminate traces of water. This operation was done 2 more times.

Solid obtained were recrystallized heating at 90° C. with 12 L of iPrOH, then suspension was cooled to 5° C. for 2 hours. Solid was filtered and washed with 3 L of iPrOH and 2 of hexanes. Solid was dried in an oven at 70° C. till constant weight, to afford 4479 g (67%) of 2-{[3-(2-oxopyrrolidin-1yl)propyl]amino}acetic acid hydrochloride salt as a white solid.

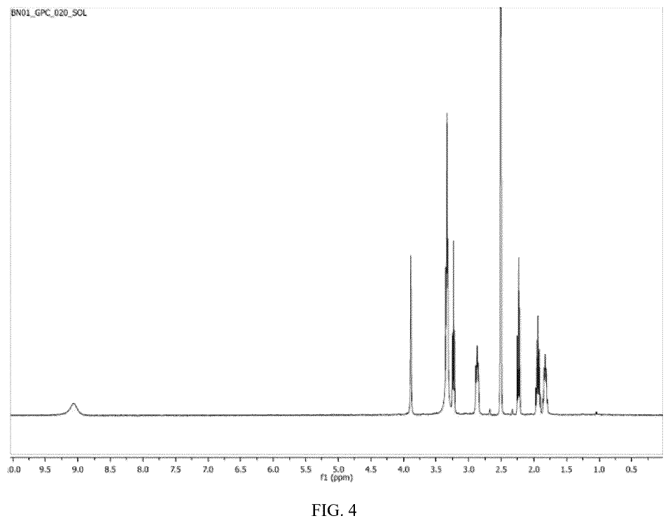

FIG. 4 shows the ¹HNMR spectra of 2-{[3-(2-oxopyrrolidin-1yl)propyl]amino}acetic acid hydrochloride in DMSO.

### Step (S4)

To a solution of 2-{N-[2-(2-fluorophenyl)ethyl]formamido}acetic acid (1300 g, 5.77 mol) in DMF (6.5 L), CDI (1122 g, 6.92 mol) was added. The resulting mixture was stirred for 1 hour. Bubbles due to CO₂ produced were observed during the first 10 min.

After 15 min the mixture was cooled at 5° C. in a water/ice bath. Then 2-[(2methylpropyl)amino]acetic acid hydrochloride (1936 g, 11.55 mol) and DBU (3450 ml, 23.08 mol) were sequentially added. After addition, internal temperature reached 10° C. The mixture was stirred for 15 min at 10° C. and then was allowed to reach 25° C.

LC-MS control after 30 min showed total conversion of the starting material.

Then 10 L of water were added and the aqueous phase was washed twice with DCM (2×2000 ml). The pH of the aqueous phase was adjusted to 2 with HCl (35%) and extracted with Ethyl acetate (3×2000 ml). Organics were collected and washed twice with brine, dried with MgSO4 and the solvent was evaporated to obtain 1560 g (80%) of desired product as brown oil. The crude will be used as it is in the next step.

### Step (S5)

To a solution of 2-(2-{N-[2-(2-fluorophenyl)ethyl]formamido}-N-(2-methylpropyl) acetamido)acetic acid (1550 g, 4.58 mol) in Acetonitrile (5600 ml) at 25° C. under nitrogen atmosphere was added in one portion Carbonyldiimidazole (890 g, 5.49 mol) and was stirred at 25° C. for 30 min. Strong evolution of CO₂ was observed.

After 30 min, reaction was added to a solution of 2-{[3-(2-oxopyrrolidin-1yl)propyl]amino}acetic acid hydrochloride (2164 g, 9.17 mol) and DBU (2735 ml, 18.32 mol) in Acetonitrile (5600 ml) under nitrogen atmosphere keeping internal temperature below 15° C. Then reaction was stirred at 25° C. for 1 hour.

At this time LC-MS showed total conversion to the desired product.

Solvent was removed and water (10 L) was added to the residue, extracted 2 times with 2000 ml of dichloromethane (DCM). Then aqueous phase was acidified with HCl (35%) till pH=2 and extracted with DCM (3×2000 ml). Organics were washed with brine, dried over magnesium sulfate and evaporated to afford 2032 g (85%) of 2-[2-(2-{N-[2-(2-fluorophenyl)ethyl]formamido}N-(2-methylpropyl)acetamido)-N-[3-(2-oxopyrrolidin-1-yl)propyl]acetamido]acetic acid as a colourless oil.

This sample will be used in the next step without further purification.

### Step (S6)

To a solution of 2-[2-(2-{N-[2-(2-fluorophenyl)ethyl]formamido}-N-(2-methylpropyl) acetamido)-N-[3-(2-oxopyrrolidin-1-yl)propyl]acetamido]acetic acid (2032 g, 3.9 mol)) in Methanol (10 L) at 25° C. was added H₂SO₄ 98% (311 ml, 5.85 mol). An exotherm transition was detected during addition, temperature raised up to 53° C. and then the reaction was heated under reflux for 12 hours.

After 12 hours LCMS showed total conversion to the desired product.

Reaction was cooled to 25° C. and poured into a solution of 1040 g of KHCO₃ (10.4 mol) in 4 L of water (pH was buffered at 7.90-7.85). Then product was extracted with DCM (3×2000 ml). Organics were combined and washed with 4 L of a solution of K₂CO₃ (25% wt/wt in water) (pH was buffered at 11.90-11.75)

Organics were washed with water (2×2000 ml) and brine (1×2000 ml), dried over magnesium sulfate and evaporated to afford 1480 g (75%) of methyl 2-[2-(2-{[2-(2fluorophenyl)ethyl]amino}-N-(2-methylpropyl)acetamido)-N-[3-(2-oxopyrrolidin-1yl)propyl]acetamido]acetate as a light brown oil.

This material will be used in the next step without further purification.

### Step (S7)

A solution of Methyl ester derivative (1480 g, 2.92 mol) in 7.4 L of methanol was bubbled at −35° C. with Ammonia (gas) (695 g, 40.88 mol), during the addition of ammonia internal temperature raised up to −3° C. This solution was left to reach 25° C. and was stirred for 24 hours.

After 24 hours LCMS showed total consumption of starting material. Solvent was removed in vacuum.

The residue was heated under reflux in 5000 ml of ethyl acetate till total solution. Then 140 g (10% wt/wt limitant reagent) of active carbon were added and suspension was stirred under ethyl acetate reflux for 1 hour.

Then suspension was filtered and washed with 2 L of ethyl acetate. Liquids were eliminated up to a volume of 6 L (5 vol of ethyl acetate+1 Kg of crude).

A white precipitate appeared. Solid was filtered and washed with 1000 ml of Ethyl acetate and 1000 ml of MTBE and dried under vacuo at 40° C. till constant weight (24 hours).

1010 g (71% yield) of N-(carbamoylmethyl)-2-(2-{[2-(2-fluorophenyl)ethyl]amino}-N-(2-methylpropyl)acetamido)-N-[3-(2-oxopyrrolidin-1yl)propyl]acetamide was obtained as a white solid.

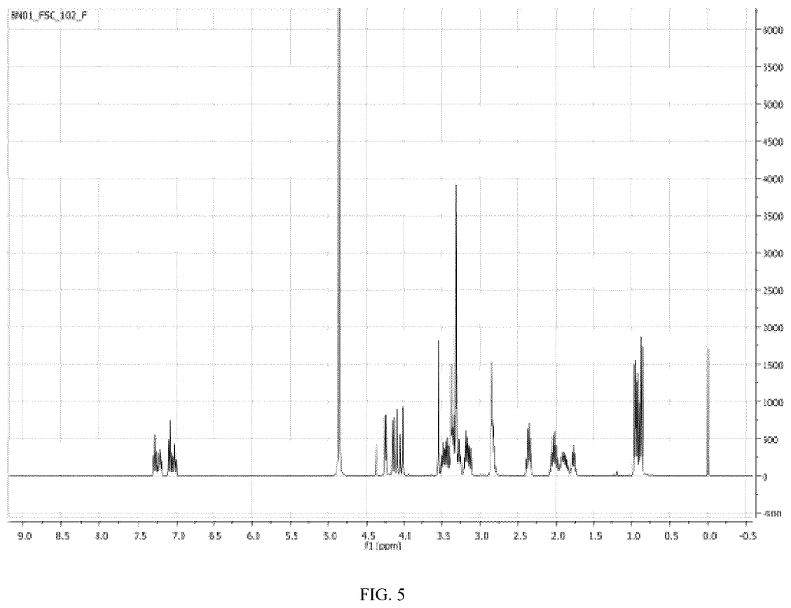

FIG. 5 shows the ¹HNMR spectra of N-(carbamoylmethyl)-2-(2-{[2-(2-fluorophenyl)ethyl]amino}-N-(2-methylpropyl)acetamido)-N-[3-(2-oxopyrrolidin-1yl)propyl]acetamide in CD₃OD.

We claim:

1. A process for synthesizing a compound of formula FI: 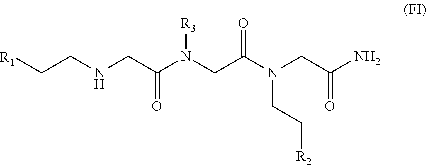
wherein:
R₁ is phenyl substituted with halogen or trifluoromethyl, and further optionally substituted with one or two substituents selected from the group consisting of halogen, (C₁-C₆)alkyl, (C₁-C₆)alkoxy, and halo(C₁-C₆)alkyl; or alternatively R₁ is pyrrolidin-1-yl;
R₂ is 2-oxo-pyrrolidin-1-ylmethyl or sulfamoylphenyl; and
R₃ is chosen from propyl, 1-methylethyl, butyl, 2-methylpropyl, pentyl, 1-methyl-butyl, 2-methylbutyl, hexyl, 4-methylpentyl, 3-methylpentyl, 2-methylpentyl, and 1-methylpentyl;
comprising the steps of:
(S1) Providing a compound CS1 of formula FCS1: 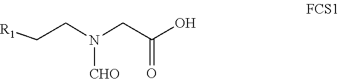
(S2) Providing a compound CS2 of formula FCS2: 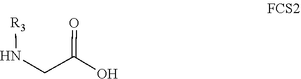
(S3) Providing a compound CS3 of formula FCS3: 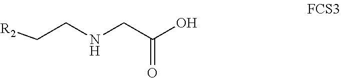
(S4) Reacting CS1 with CS2 in an organic solvent resulting in a compound CS4 of formula FCS4: 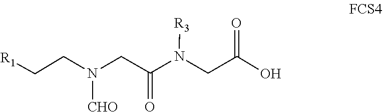
(S5) Reacting CS3 with CS4 in an organic solvent resulting in a compound CS5 of formula FCS5: 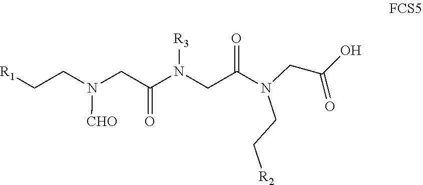
(S6) Reacting CS5 with an acid in an alcoholic solvent resulting in a compound CS6 of formula FCS6: 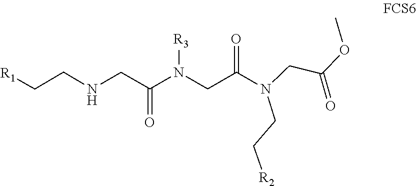
(S7) Reacting CS6 with ammonia in an organic solvent.

2. The process according to claim 1, wherein step (S1) is carried out in water at a temperature between 65° C. and 90° C.

3. The process according to claim 1, wherein step (S1) is carried out in water at a temperature between 70° C. and 85° C.

4. The process according to claim 1, wherein step (S2) is carried out in water at a temperature between 65° C. and 90° C., and then in concentrated HCl at a temperature of 80° C. or above.

5. The process according to claim 1, wherein step (S2) is carried out in water at a temperature between 70° C. and 85° C., and then in concentrated HCl at a temperature of 90° C. or above.

6. The process according to claim 1, wherein step (S3) is carried out in water at a temperature between 65° C. and 90° C., and then in concentrated HCl at a temperature of 80° C. or above.

7. The process according to claim 1, wherein step (S3) is carried out in water at a temperature between 70° C. and 85° C., and then in concentrated HCl at a temperature of 90° C. or above.

8. The process according to claim 1, wherein step (S4) is carried out at a temperature starting at 0° C. to room temperature between 20° C. and 25° C. in the presence of 2,3,4,6,7,8,9,10-octahydropyrimido[1,2-a]azepine and carbonyldiimidazole.

9. The process according to claim 1, wherein step (S5) is carried out at room temperature between 20° C. and 25° C. in the presence of 2,3,4,6,7,8,9,10-octahydropyrimido[1,2-a]azepine and carbonyldiimidazole.

10. The process according to claim 1, wherein step (S6) is carried out at reflux.

11. The process according to claim 1, wherein the compound obtained at the end of step (S7) comprises the compound of formula FI and less than 0.3% of each individual impurity.

12. A compound selected from the group consisting of:

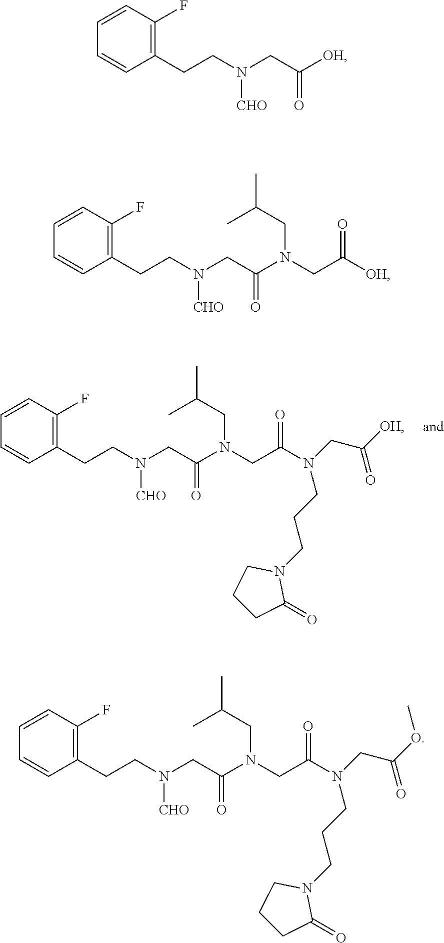 [SMILES: O=CN(CCc1ccccc1F)CC(=O)O ; CC(C)CN(CC(=O)O)C(=O)CN(C=O)CCc1ccccc1F ; CC(C)CN(CC(=O)N(CCCN1CCCC1=O)CC(=O)O)C(=O)CN(C=O)CCc1ccccc1F ; COC(=O)CN(CCCN1CCCC1=O)C(=O)CN(CC(C)C)C(=O)CN(C=O)CCc1ccccc1F]

13. The compound of claim 12, wherein the compound is:

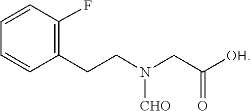 [SMILES: O=CN(CCc1ccccc1F)CC(=O)O]

14. The compound of claim 12, wherein the compound is:

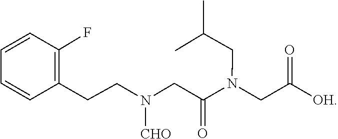 [SMILES: CC(C)CN(CC(=O)O)C(=O)CN(C=O)CCc1ccccc1F]

15. The compound of claim 12, wherein the compound is:

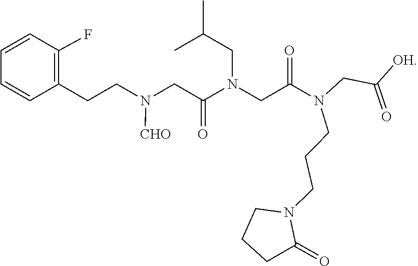 [SMILES: CC(C)CN(CC(=O)N(CCCN1CCCC1=O)CC(=O)O)C(=O)CN(C=O)CCc1ccccc1F]

16. The compound of claim 12, wherein the compound is:

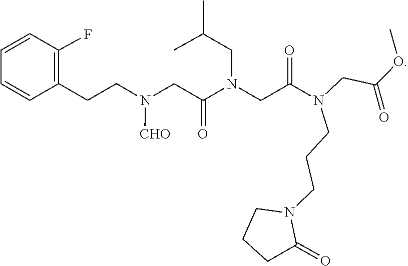 [SMILES: COC(=O)CN(CCCN1CCCC1=O)C(=O)CN(CC(C)C)C(=O)CN(C=O)CCc1ccccc1F]

17. A pharmaceutical composition comprising the compound of formula FI obtained by the process according to claim 1, and optionally one or more pharmaceutically acceptable excipient(s) comprising less than 0.3% of each individual impurity.
